(19) United States
(12) Patent Application Publication
(10) Pub. No.: US 2022/0016113 A1
(43) Pub. Date: Jan. 20, 2022

(54) ?2-ADRENOCEPTOR SUBTYPE C (ALPHA-2C) ANTAGONISTS FOR THE TREATMENT OF SLEEP APNEA

(71) Applicant: Bayer Aktiengesellschaft, Leverkusen (DE)

(72) Inventors: Martina DELBECK, Heiligenhaus (DE); Michael HAHN, Langenfeld (DE)

(73) Assignee: Bayer Aktiengesellschaft, Leverkusen (DE)

(21) Appl. No.: 17/295,774

(22) PCT Filed: Nov. 13, 2019
(86) PCT No.: PCT/EP2019/081133

§ 371 Date: May 20, 2021

(30) Foreign Application Priority Data
Nov. 20, 2018 (EP) 18207138.1

(51) Int. Cl. A61K 31/506; A61K 31/4709; A61K 45/06

(52) U.S. Cl. CPC A61K 31/506; A61K 45/06; A61K 31/4709

(57) ABSTRACT

(alpha-2C) antagonists, in particular substituted piperidinyl-pyrimidinyl-tetra hydrochinolines and piperidinyl-pyridinyl-tetrahydrochinolines of formula (I) for the use in a method for the treatment and/or prophylaxis of sleep-related breathing disorders, preferably obstructive and central sleep apneas and snoring. Formula (I) in which X is a group (A) or (B).

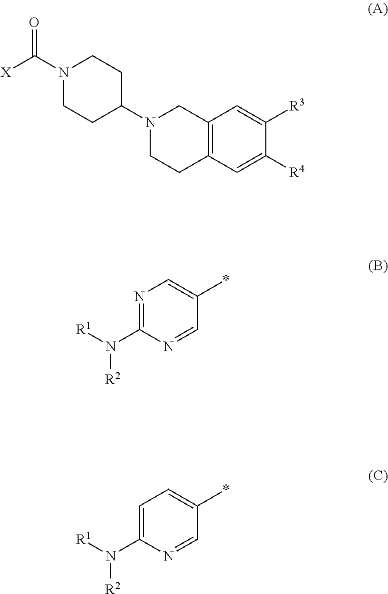

The present invention relates to α2-Adrenoceptor subtype C (alpha-2C) antagonists, in particular substituted piperidinyl-pyrimidinyl-tetrahydrochinolines and piperidinyl-pyridinyl-tetrahydrochinolines of formula (I) for the use in a method for the treatment and/or prophylaxis of sleep-related breathing disorders, preferably obstructive and central sleep apneas and snoring.

### BACKGROUND OF THE INVENTION

Obstructive sleep apnoea (OSA) is a sleep-related respiratory disorder which is characterized by repeated episodes of obstruction of the upper airways. When breathing in, the patency of the upper airways is ensured by the interaction of two opposite forces. The dilative effects of the musculature of the upper airways counteract the negative intraluminal pressure, which constricts the lumen. The active contraction of the diaphragm and the other auxiliary respiratory muscles generates a negative pressure in the airways, thus constituting the driving force for breathing. The stability of the upper respiratory tract is substantially determined by the coordination and contraction property of the dilating muscles of the upper airways.

Upper airway collapse in OSA is thought to occur at sleep onset because of the reduction of activity of several upper airway dilator muscles, which as a consequence are unable to maintain the anatomically vulnerable airway open. However, some upper airway dilator muscles, including the genioglossus muscle, which is the most important of the dilating muscles of the upper respiratory airway and which is innervated by the hypoglossal nerve, can increase activity during sleep in response to respiratory stimuli, potentially counteracting some of these changes at sleep onset. It was observed that OSA patients have apnea free intervals in which the genioglossus muscle activity is only 25-40% higher compared with sleep phases with frequent obstructive apneas (Jordan A S, White D P, Lo Y L et al., Airway dilator muscle activity and lung volume during stable breathing in obstructive sleep apnea. Sleep 2009, 32(3): 361-8). Noradrenaline is one of the most potent neuromodulators of hypoglossal motoneuron activity (Homer R. L. Neuromodulation of hypoglossal motoneurons during sleep. Respir Physiol Neurobiol 2008, 164 (1-2): 179-196). It is thought, that decreased noradrenergic drive leads to sleep-dependent decline of hypoglossal motoneuron excitability resulting in reduced upper airway dilator muscle activity, especially reduced genioglossus muscle activity.

Alpha2C adrenoceptors regulate the release of noradrenaline from central noradrenergic neurons, they are autoreceptors involved in presynaptic feedback inhibition of noradrenaline (Hein L. et al, Two functionally distinct alpha2-adrenergic receptors regulate sympathetic neurotransmission Nature 1999, 402(6758): 181-184). An increase in the activity of the motoneurons of the hypoglossal nerve through Alpha2c adrenoceptor antagonism can stabilize the upper airways and protect them from collapse and occlusion. Moreover, also snoring can be inhibited through the mechanism of stabilization of the upper respiratory airways.

For simple snoring, there is no obstruction of the upper airways. By the narrowing of the upper airways, the flow velocity of the inhaled and exhaled air increases. This together with the relaxed muscles causes fluttering of the soft tissues of the mouth and throat in the airflow. This slight vibration generated the typical snoring sounds.

Obstructive snoring (upper airway resistance syndrome, heavy snoring, hypopnea syndrome) is caused by a recurrent partial obstruction of the upper airway during sleep. This results in an increase in airway resistance and thus to an increase in work of breathing with significant intrathoracic pressure fluctuations. The negative intrathoracic pressure development during inspiration can thereby reach values as they occur as a result of a complete airway obstruction in OSA. The pathophysiological effects on the heart, circulation and sleep quality are the same as in obstructive sleep apnea. The pathogenesis is likely the same as in OSA. Obstructive snoring often provides the precursor for OSA (Hollandt J. H. et al., Upper airway resistance syndrome (UARS)-obstructive snoring. HNO 2000, 48(8): 628-634).

Central sleep apnea (CSA) occurs as a result of disturbed brain function or impaired respiratory regulation. CSA is characterized by a lack of drive to breathe during sleep, resulting in repetitive periods of insufficient or absent ventilation and compromised gas exchange. There are several manifestations of CSA. These include high altitude-induced periodic breathing, idiopathic CSA (ICSA), narcotic-induced central apnea, obesity hypoventilation syndrome (OHS), and Cheyne-Stokes breathing (CSB). While the precise precipitating mechanisms involved in the various types of CSA may vary considerably, unstable ventilatory drive during sleep is a principal underlying feature (Eckert D. J. et al., Central sleep apnea: Pathophysiology and treatment. Chest 2007, 131(2): 595-607).

US 2018/0235934 A1 describes methods for treating disorders such as obstructive sleep apnea using agents for promoting hypoglossal motoneuron excitability. As agents for promoting hypoglossal motoneuron excitability a disinhibtor and/or stimulant of central noradrenic neurons is described. In some embodiments the disinhibitor of central noradrenergic neurons is an alpha2-adrenoceptor antagonist such as yohimbine or an alpha2-adrenoceptor subtype A (alpha-2A) antagonists or alpha2-adrenoceptor subtype C (alpha-2C) antagonist. The alpha2-adrenoceptor antagonist are selected from the group consisting of Atipamezole, MK-912, RS-79948, RX 821002, [3H]2-methoxy-idazoxan and JP-1302.

Alpha2C adrenoceptors belong to the family of G-protein coupled receptors. Beside the different Alpha1-adrenoceptors three different Alpha2-adrenoceptor subtypes exist (Alpha2A, Alpha2B and Alpha2C). They are involved in the mediation of several diverse physiologic effects in different tissues upon stimulation by endogeneous catecholamines (epinephrine, norepinephrine), either derived from synapses or via the blood. Alpha2 adrenoceptors plays an important physiological role, mainly in the cardiovascular system and in the central nervous system. Alpha2A- and Alpha2C-adrenoceptors are the main autoreceptors involved in presynaptic feedback inhibition of noradrenaline in the central nervous system. The potency and affinity of noradrenaline at the Alpha2C-adrenoceptor is higher than that for the Alpha2A-adrenoceptor. The Alpha2C-adrenoceptor inhibits noradrenaline release at low endogenous concentrations of noradrenaline, while Alpha2A-adrenoceptors inhibit noradrenaline release at high endogenous noradrenaline concentrations (Uys M. M. et al. Therapeutic Potential of Selectively Targeting the α2C-Adrenoceptor in Cognition, Depression, and Schizophrenia—New Developments and Future Perspective. Frontiers in Psychiatry 2017, Aug. 14; 8:144. doi: 10.3389/fpsyt.2017.00144. eCollection 2017).

Substituted piperidinyl-pyrimidinyl-tetrahydrochinolines and piperidinyl-pyridinyl-tetrahydrochinolines as α2-Adrenoceptor subtype C (alpha-2C) antagonists as well as their preparation and the use thereof as a medicament are known from WO 2015/091414 A1 and in WO 2015/091417 A1 where the compounds are disclosed as useful for the treatment and/or prophylaxis of primary and secondary forms of diabetic microangiopathies, diabetic wound healing, diabetic ulcers on the extremities, in particular for promoting wound healing of diabetic foot ulcers, diabetic retinopathy, diabetic nephropathy, diabetic erectile dysfunction, diabetic heart failure, diabetic coronary microvascular heart disorders, peripheral and cardial vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies. There is nothing disclosed about the use of these compounds in the treatment of sleep-related breathing disorders, preferably obstructive and central sleep apneas and snoring.

The current gold standard treatment for patients with OSA is continuous positive airway pressure (CPAP). The positive airflow pressure that is generated by an airflow turbine pump splints open the upper airway, reversing all potential causes of pharyngeal collapse, thereby preventing hypopneas, apneas and sleep fragmentation. Unfortunately, up to 50% of all patients with OSA do not tolerate CPAP in the long-term (M. Kohler, D. Smith, V. Tippett et al., Thorax 2010 65(9):829-32: Predictors of long-term compliance with continuous positive airway pressure). Therefore, there is still the need to find effective therapeutic agents for the treatment and/or prophalxis of sleep-related breathing disorders such as obstructive sleep apnea. Therefore the object of the present invention is to provide an effective therapeutic agent for the treatment and/or prophalxis of sleep-related breathing disorders, for example of obstructive sleep apnea, central sleep apnea and snoring.

Surprisingly, it has now been found that substituted piperidinyl-pyrimidinyl-tetrahydrochinoline and piperidinyl-pyridinyl-tetrahydrochinoline of formula (I) of the present invention inhibit upper airway collapsibility and are thus suitable for the production of medicaments for the use in the treatment and/or prophylaxis of sleep-related breathing disorders, preferably obstructive and central sleep apneas and snoring.

The present invention relates to compounds of formula (I)

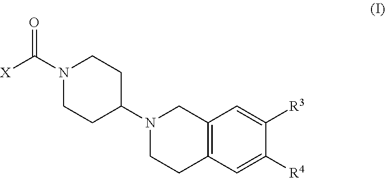

in which

X is a group

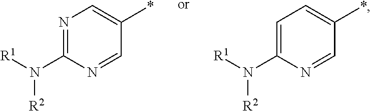

R¹ represents C₁-C₆-alkyl or C₃-C₅-cycloalkyl,
where alkyl is substituted by 1 to 2 substituents independently of one another selected from the group consisting of hydroxy, C₁-C₄-alkoxy and haloalkoxy

and

R² represents hydrogen or C₁-C₄-alkyl,

or

R¹ and R² together with the nitrogen atom to which they are attached form a 4- to 7-membered N-heterocycle,

where the N-heterocycle may be substituted by 1 to 3 substituents independently of one another selected from the group consisting of oxo, hydroxy, monofluoromethyl, difluoromethyl, trifluoromethyl, hydroxycarbonyl, tert-butoxycarbonyl, aminocarbonyl, C₁-C₄-alkyl, C₁-C₄-alkoxy, C₁-C₄-alkoxy-C₁-C₄-alkyl, halogen and hydroxyalkyl,

or

where the N-heterocycle may have two substituents which, together with the carbon atom of the N-heterocycle to which they are jointly attached, form a 4- to 6-membered heterocycle,
where this heterocycle for its part may be substituted by 1 to 3 substituents independently of one another selected from the group consisting of oxo, methyl and ethyl,

R³ represents hydrogen, fluorine, methoxy or ethoxy

and

R⁴ represents hydrogen, fluorine, methoxy or ethoxy,

and the salts thereof, the solvates thereof and the solvates of the salts thereof,

for the use in a method for the treatment and/or prophylaxis of sleep-related breathing disorders, preferably obstructive and central sleep apneas and snoring.

In the context of the present invention, unless specified otherwise, the substituents are defined as follows:

Alkyl per se and “Alk” and “alkyl” in alkoxy, alkoxyalkyl, alkylamino and alkoxycarbonyl represent a straight-chain or branched alkyl radical having 1 to 6 carbon atoms, preferably 1 to 4 carbon atoms, by way of example and with preference methyl, ethyl, n-propyl, isopropyl, n-butyl, sec-butyl, tert-butyl, n-pentyl and n-hexyl.

Alkoxy, by way of example and with preference, represents methoxy, ethoxy, n-propoxy, isopropoxy, n-butoxy and tert-butoxy.

Alkoxyalkyl, by way of example and with preference, represents methoxymethyl, ethoxymethyl, n-propoxymethyl, isopropoxymethyl, n-butoxymethyl, tert-butoxymethyl, methoxyethyl, ethoxyethyl, n-propoxyethyl, isopropoxyethyl, n-butoxyethyl and tert-butoxyethyl.

N-Heterocycle in the definition of the radicals R¹ and R² represents a saturated and partially unsaturated monocyclic radical having 4 to 7 ring atoms having a nitrogen heteroatom and up to 3 further heteroatoms and/or hetero groups from the group consisting of S, O, N, SO and SO₂, where a nitrogen atom may also form an N-oxide, by way of example and with preference azetidine, pyrrolidine, piperidine, azepane, piperazine, morpholine, thiomorpholine, 1-oxidothiomorpholine and 1,1-dioxidothiomorpholine, particularly preferably azetidine, pyrrolidine, morpholine and 1,1-dioxidothiomorpholine.

Heterocycle in the definition of the radicals R¹ and R², having a joint carbon atom with the N-heterocycle to which it is attached, represents a saturated and partially unsaturated monocyclic radical having 4 to 6 ring atoms and up to 4 heteroatoms and/or hetero groups from the group consisting of S, O, N, SO and SO₂, where a nitrogen atom may also form an N-oxide, by way of example and with preference azetidine, oxetane, thietane, pyrrolidine, tetrahydrofuran, piperidine, morpholine, thiomorpholine, piperazine, tetrahydropyran and 1,1-dioxidothietane, particularly preferably azetidine and oxetane and even more preferably oxetane.

Halogen represents fluorine, chlorine, bromine and iodine, preferably fluorine and chlorine.

The term “hydroxy”, as employed herein as such or as part of another group, refers to a OH group.

The expression “compounds of the invention” as employed herein refers to the compounds of formula I.

Pharmaceutically acceptable salts, e.g. acid addition salts, with both organic and inorganic acids, are known in the field of pharmaceuticals. Representative examples of pharmaceutically acceptable acid addition salts include, but are not limited to, chlorides, bromides, sulfates, nitrates, phosphates, sulfonates, methane sulfonates, formates, tartrates, maleates, citrates, benzoates, salicylates, ascorbates, acetates and oxalates.

Hydrates or solvates are designated according to the invention as those forms of the compounds of the formula (I) which in the solid or liquid state form a molecular compound or a complex by hydration with water or coordination with solvent molecules. Examples of hydrates are sesquihydrates, monohydrates, dihydrates or trihydrates. Equally, the hydrates or solvates of salts of the compounds according to the invention are also suitable.

Pharmaceutically acceptable esters, when applicable, may be prepared by known methods using pharmaceutically acceptable acids that are conventional in the field of pharmaceuticals and that retain the pharmacological properties of the free form. Nonlimiting examples of these esters include esters of aliphatic or aromatic alcohols. Representative examples of pharmaceutically acceptable esters include, but are not limited to, methyl, ethyl, n-propyl, iso-propyl, n-butyl, iso-butyl, sec-butyl, tert-butyl, and benzylesters.

The invention includes within its scope all the possible geometric isomers, e.g. Z and E isomers (cis and trans isomers), of the compounds as well as all the possible optical isomers, e.g. diastereomers and enantiomers, of the compounds. Furthermore, the invention includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g. enantiomers, from the mixture thereof, conventional resolution methods, e.g. fractional crystallization, may be used.

A particular embodiment of the present invention relates to compounds of the formula (I) in which
R¹ represents C₁-C₆-alkyl or C₃-C₅-cycloalkyl,

where alkyl is substituted by 1 to 2 substituents independently of one another selected from the group consisting of hydroxy and C₁-C₄-alkoxy,

and
R² represents hydrogen or C₁-C₄-alkyl,

or
R¹ and R² together with the nitrogen atom to which they are attached form a 4- to 7-membered N-heterocycle,
   where the N-heterocycle may be substituted by 1 to 3 substituents independently of one another selected from the group consisting of oxo, hydroxy, monofluoromethyl, difluoromethyl, trifluoromethyl, hydroxycarbonyl, tert-butoxycarbonyl, aminocarbonyl, C₁-C₄-alkoxy and halogen,
   or
   where the N-heterocycle may have two substituents which, together with the carbon atom of the N-heterocycle to which they are jointly attached, form a 4- to 6-membered heterocycle,
      where this heterocycle for its part may be substituted by 1 to 3 substituents independently of one another selected from the group consisting of oxo, methyl and ethyl,
R³ represents hydrogen, fluorine, methoxy or ethoxy,

and
R⁴ represents hydrogen, fluorine, methoxy or ethoxy,

and the salts thereof, the solvates thereof and the solvates of the salts thereof, for the use in a method for the treatment and/or prophylaxis of sleep-related breathing disorders.

A further particular embodiment of the present invention relates to compounds of the formula (I) in which
R¹ represents C₂-C₆-alkyl,
   where alkyl is substituted by a substituent selected from the group consisting of hydroxy, methoxy and ethoxy,

and
R² represents hydrogen,

or
R¹ and R² together with the nitrogen atom to which they are attached form an azetidine, pyrrolidine, piperidine, azepane, piperazine, morpholine, thiomorpholine, 1-oxidothiomorpholine or 1,1-dioxidothiomorpholine,
   where azetidine, pyrrolidine, piperidine, azepane, piperazine, morpholine, thiomorpholine, 1-oxidothiomorpholine and 1,1-dioxidothiomorpholine may be substituted by 1 to 2 substituents independently of one another selected from the group consisting of hydroxy, trifluoromethyl, hydroxycarbonyl, C₁-C₃-alkyl, methoxy and methoxymethyl,
   or
   where azetidine, pyrrolidine, piperidine, azepane, piperazine and morpholine may have two substituents which, together with the carbon atom of the azetidine, pyrrolidine, piperidine, azepane, piperazine or morpholine to which they are where this azetidine, oxetane or 1,1-dioxidothietane for its part may be substituted by 1 to 2 substituents independently of one another selected from the group consisting of methyl and ethyl,
R³ represents hydrogen

and
R⁴ represents hydrogen, fluorine or methoxy,

or
R³ represents hydrogen, fluorine or methoxy

and
R⁴ represents hydrogen,

and the salts thereof, the solvates thereof and the solvates of the salts thereof, for the use in a method for the treatment and/or prophylaxis of sleep-related breathing disorders.

A further particular embodiment of the present invention relates to compounds of the formula (I) in which
R¹ represents C₂-C₄-alkyl,
   where alkyl is substituted by a substituent selected from the group consisting of hydroxy and methoxy,

and
R² represents hydrogen,

or
R¹ and R² together with the nitrogen atom to which they are attached form an azetidine, pyrrolidine, morpholine or 1,1-dioxidothiomorpholine,
   where azetidine, pyrrolidine, morpholine or 1,1-dioxidothiomorpholine may be substituted by 1 to 2 substituents selected independently from the group consisting of hydroxycarbonyl, methyl, trifluoromethyl, methoxy and

or
R¹ and R² together with the nitrogen atom to which they are attached form an azetidine,
   where the azetidine may have two substituents which, together with the carbon atom of the azetidine to which they are jointly attached, form an oxetane or 1,1-dioxidothietane,
R³ represents hydrogen, fluorine or methoxy

and
R⁴ represents hydrogen,

or
R³ represents hydrogen,

and
R⁴ represents hydrogen, fluorine or methoxy,

and the salts thereof, the solvates thereof and the solvates of the salts thereof, for the use in a method for the treatment and/or prophylaxis of sleep-related breathing disorders.

A further particular embodiment of the present invention relates to compounds of the formula (I) in which
R¹ represents C₂-C₄-alkyl,
   where alkyl is substituted by a substituent selected from the group consisting of hydroxy and methoxy,

and
R² represents hydrogen,

or
R¹ and R² together with the nitrogen atom to which they are attached form an azetidine, pyrrolidine, morpholine or 1,1-dioxidothiomorpholine,
   where azetidine, pyrrolidine, morpholine or 1,1-dioxidothiomorpholine may be substituted by 1 to 2 substituents selected independently from the group consisting of hydroxycarbonyl and methyl, trifluoromethyl, methoxy and methoxymethyl,
or
R¹ and R² together with the nitrogen atom to which they are attached form an azetidine,
   where the azetidine may have two substituents which, together with the carbon atom of the azetidine to which they are jointly attached, form an oxetane, or 1,1-dioxidothietane
R³ represents hydrogen or fluorine,

and
R⁴ represents hydrogen, fluorine or methoxy,

and the salts thereof, the solvates thereof and the solvates of the salts thereof, for the use in a method for the treatment and/or prophylaxis of sleep-related breathing disorders.

A further particular preferred embodiment of the present invention relates to compounds of the formula (I) in which
X is a group

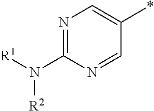

R¹ represents C₂-C₄-alkyl,
   where alkyl is substituted by a substituent selected from the group consisting of hydroxy and methoxy,

and
R² represents hydrogen,

or
R¹ and R² together with the nitrogen atom to which they are attached form an azetidine, pyrrolidine, morpholine or 1,1-dioxidothiomorpholine,
   where azetidine, pyrrolidine, morpholine or 1,1-dioxidothiomorpholine may be substituted by 1 to 2 substituents selected independently from the group consisting of hydroxycarbonyl and methyl,

or
R¹ and R² together with the nitrogen atom to which they are attached form an azetidine,
   where the azetidine may have two substituents which, together with the carbon atom of the azetidine to which they are jointly attached, form an oxetane,
R³ represents hydrogen, fluor or methoxy,

and
R⁴ represents hydrogen,

or
R³ represents hydrogen,

and
R⁴ represents hydrogen, fluor or methoxy,

and the salts thereof, the solvates thereof and the solvates of the salts thereof, for the use in a method for the treatment and/or prophylaxis of sleep-related breathing disorders.

A further particular preferred embodiment of the present invention relates to compounds of the formula (I) in which
X is a group

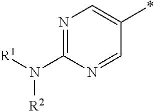

R¹ and R² together with the nitrogen atom to which they are attached form an azetidine,
   where the azetidine has two substituents which, together with the carbon atom of the azetidine to which they are jointly attached, form an oxetane,
R³ represents hydrogen,

and
R⁴ represents hydrogen,

and the salts thereof, the solvates thereof and the solvates of the salts thereof, for the use in a method for the treatment and/or prophylaxis of sleep-related breathing disorders.

Preference is also given to compounds of the formula (I) in which R² represents hydrogen.

Preference is also given to compounds of the formula (I) in which R¹ and R² together with the nitrogen atom to which they are attached represent 2-oxa-6-azaspiro[3.3]hept-6-yl.

Preference is also given to compounds of the formula (I) in which R¹ and R² together with the nitrogen atom to which they are attached represent 1,1-dioxidothiomorpholin-4-yl.

Preference is also given to compounds of the formula (I) in which R³ represents hydrogen.

Preference is also given to compounds of the formula (I) in which R⁴ represents hydrogen.

Preference is also given to compounds of the formula (I) in which R³ and R⁴ represent hydrogen.

In a preferred embodiment the present invention relates to a compound of the formula (I) selected from the group consisting of:

[4-(3,4-Dihydroisoquinolin-2(1H)-yl)piperidin-1-yl]{2-[(2-methoxyethyl)amino]pyrimidin-5-yl}methanone, [4-(7-Fluoro-3,4-dihydroisoquinolin-2(1H)-yl)piperidin-1-yl]{2-[(1-methoxybutan-2-yl)amino]pyrimidin-5-yl}methanone, [4-(6-Fluoro-3,4-dihydroisoquinolin-2(1H)-yl)piperidin-1-yl]{2-[(1-methoxybutan-2-yl)amino]pyrimidin-5-yl}methanone, {2-[(1-Methoxybutan-2-yl)amino]pyrimidin-5-yl}[4-(7-methoxy-3,4-dihydroisoquinolin-2(1H)-yl)piperidin-1-yl]methanone, {2-[(1-Methoxybutan-2-yl)amino]pyrimidin-5-yl}[4-(6-methoxy-3,4-dihydroisoquinolin-2(1H)-yl)piperidin-1-yl]methanone, [4-(3,4-Dihydroisoquinolin-2(1H)-yl)piperidin-1-yl]{2-[(1-methoxybutan-2-yl)amino]pyrimidin-5-yl}methanone, [4-(3,4-Dihydroisoquinolin-2(1H)-yl)piperidin-1-yl]{2-[(1-hydroxybutan-2-yl)amino]pyrimidin-5-yl}methanone, [4-(3,4-Dihydroisoquinolin-2(1H)-yl)piperidin-1-yl][2-(2-oxa-6-azaspiro[3.3]hept-6-yl)pyrimidin-5-yl]methanone, [4-(7-Fluoro-3,4-dihydroisoquinolin-2(1H)-yl)piperidin-1-yl][2-(2-oxa-6-azaspiro[3.3]hept-6-yl)pyrimidin-5-yl]methanone, [4-(6-Methoxy-3,4-dihydroisoquinolin-2(1H)-yl)piperidin-1-yl][2-(2-oxa-6-azaspiro[3.3]hept-6-yl)pyrimidin-5-yl]methanone, 1-(5-{[4-(3,4-Dihydroisoquinolin-2(1H)-yl)piperidin-1-yl]carbonyl}pyrimidin-2-yl)-D-proline hydrochloride, [4-(3,4-Dihydroisoquinolin-2(1H)-yl)piperidin-1-yl][2-(1,1-dioxidothiomoipholin-4-yl)pyrimidin-5-yl]methanone, [4-(3,4-Dihydroisoquinolin-2(1H)-yl)piperidin-1-yl][2-(2,6-dimethylmorpholin-4-yl)pyrimidin-5-yl]methanone, [4-(3,4-Dihydroisoquinolin-2(1H)-yl)piperidin-1-yl][2-(2,6-dimethylmorpholin-4-yl)pyrimidin-5-yl]methanone, [4-(3,4-Dihydroisoquinolin-2(1H)-yl)piperidin-1-yl][2-(2,2-dimethylmorpholin-4-yl)pyrimidin-5-yl]methanone, [4-(3,4-Dihydroisoquinolin-2(1H)-yl)piperidin-1-yl]{6-[(2-methoxyethyl)amino]pyridin-3-yl}methanone, [4-(7-Fluoro-3,4-dihydroisoquinolin-2(1H)-yl)piperidin-1-yl]{6-[(2-methoxyethyl)amino]pyridin-3-yl}methanone, [4-(3,4-Dihydroisoquinolin-2(1H)-yl)piperidin-1-yl][6-(morpholin-4-yl)pyridin-3-yl]methanone, [4-(3,4-Dihydroisoquinolin-2(1H)-yl)piperidin-1-yl][6-(1,1-dioxidothiomorpholin-4-yl)pyridin-3-yl]methanone, [4-(3,4-Dihydroisoquinolin-2(1H)-yl)piperidin-1-yl]{6-[(2R)-2-(methoxymethyl)pyrrolidin-1-yl]pyridin-3-yl}methanone, 1-(5-{[4-(3,4-Dihydroisoquinolin-2(1H)-yl)piperidin-1-yl]carbonyl}pyridin-2-yl)-D-pro line, [4-(3,4-Dihydroisoquinolin-2(1H)-yl)piperidin-1-yl](6-{[(2 S)-1-hydroxybutan-2-yl]amino}pyridin-3-yl)methanone, [4-(6-Fluoro-3,4-dihydroisoquinolin-2(1H)-yl)piperidin-1-yl][6-(3-methoxypyrrolidin-1-yl)pyridin-3-yl]methanone, [4-(6-Methoxy-3,4-dihydroisoquinolin-2(1H)-yl)piperidin-1-yl][6-(3-methoxypyrrolidin-1-yl)pyridin-3-yl]methanone, [4-(3,4-Dihydroisoquinolin-2(1H)-yl)piperidin-1-yl][6-(2-oxa-6-azaspiro[3.3]hept-6-yl)pyridin-3-yl]methanone, [4-(3,4-Dihydroisoquinolin-2(1H)-yl)piperidin-1-yl][6-(2,6-dimethylmorpholin-4-yl)pyridin-3-yl]methanone, [4-(3,4-Dihydroisoquinolin-2(1H)-yl)piperidin-1-yl][6-(2,6-dimethylmorpholin-4-yl)pyridin-3-yl]methanone, [4-(3,4-Dihydroisoquinolin-2(1H)-yl)piperidin-1-yl][6-(2,2-dimethylmorpholin-4-yl)pyridin-3-yl]methanone, [4-(3,4-Dihydroisoquinolin-2(1H)-yl)piperidin-1-yl]{6-[2-(trifluoromethyl)moipholin-4-yl]pyridin-3-yl}methanone, [4-(3,4-Dihydroisoquinolin-2(1H)-yl)piperidin-1-yl][6-(2,2-dioxido-2-thia-6-azaspiro[3.3]hept-6-yl)pyridin-3-yl]methanone and also their salts, solvates, and solvates of the salts, for the use in a method for the treatment and/or prophylaxis of sleep-related breathing disorders, preferably obstructive and central sleep apneas and snoring.

In a more preferred embodiment the present invention relates to a compound [4-(3,4-Dihydroisoquinolin-2(1H)-yl)piperidin-1-yl][2-(2-oxa-6-azaspiro[3.3]hept-6-yl)pyrimidin-5-yl]methanone,

and also its salts, solvates, and solvates of the salts, for the use in a method for the treatment and/or prophylaxis of sleep-related breathing disorders, preferably obstructive and central sleep apneas and snoring.

The compounds of formula (I), their production and their action as alpha2C antagonists for use in a method for the treatment and/or prophylaxis of primary and secondary forms of diabetic microangiopathies, diabetic wound healing, diabetic ulcers on the extremities, in particular for promoting wound healing of diabetic foot ulcers, diabetic retinopathy, diabetic nephropathy, diabetic erectile dysfunction, diabetic heart failure, diabetic coronary microvascular heart disorders, peripheral and cardial vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies are disclosed in WO 2015/091414 and in WO 2015/091417 in general and especially the compounds specifically are an explicit part of the description of the present invention and are hereby incorporated by reference.

The term effective amount as used herein refers to an amount of a compound of formula (I) that is effective for treatment and/or prophylaxis of sleep-related breathing disorders, preferably obstructive and central sleep apneas and snoring.

The present invention relates to (alpha-2C) antagonists, in particular the substituted piperidinyl-pyrimidinyl-tetrahydrochinolines and piperidinyl-pyridinyl-tetrahydrochinolines of formula (I), for the use in a method for the treatment and/or prophylaxis of sleep-related breathing disorders, preferably obstructive and central sleep apneas and snoring.

The present invention further relates to the use of compounds of formula (I) for the manufacture of medicaments for the treatment and/or prophylaxis of sleep-related breathing disorders, preferably obstructive and central sleep apneas and snoring.

A further subject of the present invention is the use of a combination of one or more compounds of the formula (I) with one or more other active compounds in a method for the treatment and/or prophylaxis sleep-related breathing disorders, preferably obstructive and central sleep apneas and snoring.

A further subject of the present invention is a pharmaceutical composition comprising at least one compounds of the formula (I) in combination with one or more inert non-toxic pharmaceutically suitable excipients for use in a method for the treatment and/or prophylaxis sleep-related breathing disorders, preferably obstructive and central sleep apneas and snoring.

The present invention further relates to pharmaceutical composition comprising a combination with one or more other active compounds in combination with one or more inert non-toxic pharmaceutically suitable excipients for use in a method for the treatment and/or prophylaxis sleep-related breathing disorders, preferably obstructive and central sleep apneas and snoring.

The present invention is also directed to a method for the treatment and/or prophylaxis of sleep-related breathing disorders, by administering systemically and/or locally a therapeutically effective amount of at least one compound of formula (I) or a medicament comprising at least one compound od formula (I) in combination with a inert, non-toxic, pharmaceutically acceptable additive.

A further subject of the present invention is a combination of one or more compounds of the formula (I) with one or more other active compounds for use in a method for the treatment and/or prophylaxis sleep-related breathing disorders, preferably obstructive and central sleep apneas and snoring.

Substituted piperidinyl-pyrimidinyl-tetrahydrochinolines and piperidinyl-pyridinyl-tetrahydrochinolines of formula (I) according to the invention can be used alone or, if required, in combination with one or more other pharmacologically active substances, provided that this combination does not lead to undesirable and unacceptable side effects. Preferred examples of combination suitable for the purpose to treat sleep-related breathing disorders, preferably obstructive and central sleep apneas and snoring, include:
respiratory stimulants such as, by way of example and with preference, theophylline, doxapram, nikethamide or caffeine;
psychostimulants such as, by way of example and with preference, modafinil or armodafinil;
amphetamines and amphetamine derivatives such as, by way of example and with preference, amphetamine, metamphetamine or methylphenidate;
serotonin reuptake inhibitors such as, by way of example and with preference, fluoxetine, paroxetine, citalopram, escitalopram, sertraline, fluvoxamine or trazodone;
serotonin precursors such as, by way of example and with preference, L-tryptophan;
selective serotonin noradrenaline reuptake inhibitors such as, by way of example and with preference, venlafaxine or duloxetine;
noradrenergic and specific serotonergic antidepressants such as, by way of example and with preference, mirtazapine;
selective noradrenaline reuptake inhibitors such as, by way of example and with preference, reboxetine or atomoxetine;
tricyclic antidepressants such as, by way of example and with preference, amitriptyline, protriptyline, doxepine, trimipramine, imipramine, clomipramine or desipramine;
muscarinic receptor antagonists, by way of example and with preference oxybutynin;
GABA agonists such as, by way of example and with preference, baclofen;
glucocorticoids such as, by way of example and with preference, fluticasone, budesonide, beclometasone, mometasone, tixocortol or triamcinolone;
cannabinoid receptor agonists;
carboanhydrase inhibitors such as, by way of example and with preference, acetazolamide, methazolamide or diclofenamide;
opioid and benzodiazepine receptor antagonists such as, by way of example and with preference, flumazenil, naloxone or naltrexone;
cholinesterase inhibitors such as, by way of example and with preference, neostigmine, pyridostigmine, physostigmine donepezil, galantamine or rivastigmine;
appetite suppressants such as, by way of example and with preference, sibutramin, opiramate, phentermine, lipase inhibitors or cannabinoid receptor antagonists;
mineralocorticoid receptor antagonists.

A preferred subject of the present invention is a combination of one or more compounds of the formula (I) with one or more other active compounds selected from the groups consisting of muscarinic receptor antagonists, mineralocorticoid receptor antagonists, diuretics, corticosteroids for use in a method for the treatment and/or prophylaxis sleep-related breathing disorders, preferably obstructive and central sleep apneas and snoring.

In a preferred embodiment of the invention, the compounds of the invention are administered in combination with a muscarinic receptor antagonist, by way of example and with preference oxybutynin.

In a preferred embodiment of the invention, the compounds of the invention are administered in combination with a mineralocorticoid receptor antagonist, by way of example and with preference spironolactone, eplerenone or finerenone.

In a preferred embodiment of the invention, the compounds of the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

In a preferred embodiment of the invention, the compounds of the invention are administered in combination with a corticosteroid, by way of example and with preference prednisone, prednisolone, methylprednisolone, triamcinolone, dexamethasone, betamethasone, beclomethasone, flunisolide, budesonide or fluticasone.

If required, aryl piperazines of formula (I) according to the invention can also be employed in conjunction with the use of one or more medical technical devices or auxiliaries, provided this does not lead to unwanted and unacceptable side-effects. Medical devices and auxiliaries suitable for such a combined application are, by way of example and with preference:
devices for positive airway pressure ventilation such as, by way of example and with preference, CPAP (continuous positive airway pressure) devices, BiPAP (bilevel positive airway pressure) devices and IPPV (intermittent positive pressure ventilation) devices;
neurostimulators of the Nervus hypoglossus;
oral appliances such as, by way of example and with preference, mandibular advancement devices;
nasal disposable valves;
nasal stents.

Substituted piperidinyl-pyrimidinyl-tetrahydrochinolines and piperidinyl-pyridinyl-tetrahydrochinolines of formula (I) according to the invention can act systemically and/or locally. For this purpose, they can be administered in a suitable manner, for example by the oral, parenteral, pulmonal, intrapulmonal (inhalative), nasal, intranasal, pharyngeal, lingual, sublingual, buccal, rectal, dermal, transdermal, conjunctival or otic route, or as an implant or stent.

A further subject of the present invention is a pharmaceutical composition comprising a compound of the formula (I) for the systemically and/or locally administration by the oral, parenteral, pulmonal, intrapulmonal (inhalative), nasal, intranasal, pharyngeal, lingual, sublingual, buccal, rectal, dermal, transdermal, conjunctival or otic route, or as an implant or stent. The preferred administration is the oral route.

For these administration routes, the compounds according to the invention can be administered in suitable administration forms.

For oral administration, administration forms which function according to the state of the art, releasing the compounds according to the invention rapidly and/or in a modified manner, which contain the compounds according to the invention in crystalline and/or amorphized and/or dissolved form, such as for example tablets (uncoated or coated tablets, for example with gastric juice-resistant or delayed dissolution or insoluble coatings, which control the release of the compound according to the invention), tablets rapidly disintegrating in the oral cavity or films/wafers, films/lyophilisates, capsules (for example hard or soft gelatine capsules), dragees, granules, pellets, powders, emulsions, suspensions, aerosols or solutions are suitable.

Parenteral administration can be effected omitting an absorption step (e.g. intravenous, intra-arterial, intracardial, intraspinal or intralumbar administration) or involving absorption (e.g. intra-muscular, subcutaneous, intracutaneous, percutaneous or intraperitoneal administration). Suitable administration forms for parenteral administration include injection and infusion preparations in the form of solutions, suspensions, emulsions, lyophilisates or sterile powders.

For the other administration routes, for example inhalation formulations (including powder inhalers and nebulisers), nasal drops, solutions or sprays, tablets for lingual, sublingual or buccal administration, tablets, films/wafers or capsules, suppositories, oral or ophthalmic preparations, vaginal capsules, aqueous suspensions (lotions, shakable mixtures), lipophilic suspensions, ointments, creams, transdermal therapeutic systems (e.g. plasters), milk, pastes, foams, dusting powders, implants or stents are suitable.

Oral or parenteral administration, in particular oral and intravenous administration, are preferred.

The compounds according to the invention can be converted into the stated administration forms. This can be effected in a manner known per se by mixing with inert, non-toxic, pharmaceutically suitable additives. These additives include carriers (for example microcrystalline cellulose, lactose, mannitol), solvents (e.g. liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (e.g. antioxidants such as for example ascorbic acid), colourants (e.g. inorganic pigments such as for example iron oxides) and flavour or odour correctors.

In general, to achieve effective results in parenteral administration it has been found advantageous to administer quantities of about 0.001 to 10 mg/kg, preferably about 0.01 to 1 mg/kg body weight. In oral administration, the dosage is about 0.01 bis 100 mg/kg, preferably about 0.01 to 20 mg/kg and quite especially preferably 0.1 to 15 mg/kg body weight.

Nonetheless it can sometimes be necessary to deviate from the said quantities, namely depending on body weight, administration route, individual response to the active substance, nature of the preparation and time or interval at which administration takes place. Thus in some cases it can be sufficient to manage with less than the aforesaid minimum quantity, while in other cases the stated upper limit must be exceeded. In the event of administration of larger quantities, it may be advisable to divide these into several individual administrations through the day.

The following practical examples illustrate the invention. The invention is not limited to the examples.

### EXAMPLES

A. Experimental Methods

Advantageous pharmacological properties of the compounds of the present invention can be determined by the following methods.

The therapeutic potential of the compounds of formula (I) according to the present invention in sleep apnea has been assessed preclinically in a pig model of obstructive sleep apnea (OSA).

Using negative pressure, it is possible to induce collapse and thus obstruction of the upper respiratory tract in anaesthetized, spontaneously breathing pigs (Wirth K. J. et al., Sleep 36(5) (2013) pp. 699-708).

German Landrace pigs are used for the model. The pigs are anaesthetized and tracheotomized. Two tracheal cannulas are inserted into the trachea, one into the rostral part and the other into the caudal part of the trachea. Using a connection piece, the rostral cannula is connected to a tube to the negative pressure device and to the distal tracheal cannula. The distal tracheal cannula is additionally connected to a tube with an open end to atmosphere via a connection piece that served for free tracheal breathing, circumventing the upper airway. By appropriate opening and clamping of those tubes breathing can be switched from nasal breathing to breathing through the caudal tracheal cannula, circumventing the upper airway, and the (isolated) upper airway can be connected to the negative pressure device, causing airflow in the inspiratory direction.

At certain points in time, the collapsibility of the upper respiratory tract is tested by having the pig breathe via the caudal cannula and applying negative pressures of −50, −100 and −150 cm water head (cm H₂O) to the upper respiratory tract. This causes the upper respiratory tract to collapse, which manifests itself in an interruption of the airflow and a pressure drop in the tube system. This test is conducted prior to the administration of the test substance and at certain intervals after the administration of the test substance. An appropriately effective test substance can prevent this collapse of the respiratory tract in the inspiratory phase.

In this OSA pig model, systemic application of the α2-Adrenoceptor subtype C (alpha-2C) antagonists of [4-(3,4-Dihydroisoquinolin-2(1H)-yl)piperidin-1-yl][2-(2-oxa-6-azaspiro[3.3]hept-6-yl)pyrimidin-5-yl]methanone with i.v. bolus injection of 0.007 mg/kg followed by an i.v. infusion of 0.0025 mg/kg/h for four hours inhibited upper airway collapsibility at a negative pressure of −50 cm water head for up to two hours and at a negative pressure of −100 cm water head for up to 90 minutes. [4-(3,4-Dihydroisoquinolin-2(1H)-yl)piperidin-1-yl][2-(2-oxa-6-azaspiro[3.3]hept-6-yl)pyrimidin-5-yl]methanone with i.v. bolus injection of 0.07 mg/kg followed by an i.v. infusion of 0.025 mg/kg/h for four hours inhibited upper airway collapsibility at a negative pressure of −150 cm water head for up to two hours and at the negative pressures of −50 and −100 cm water head for up to four hours.

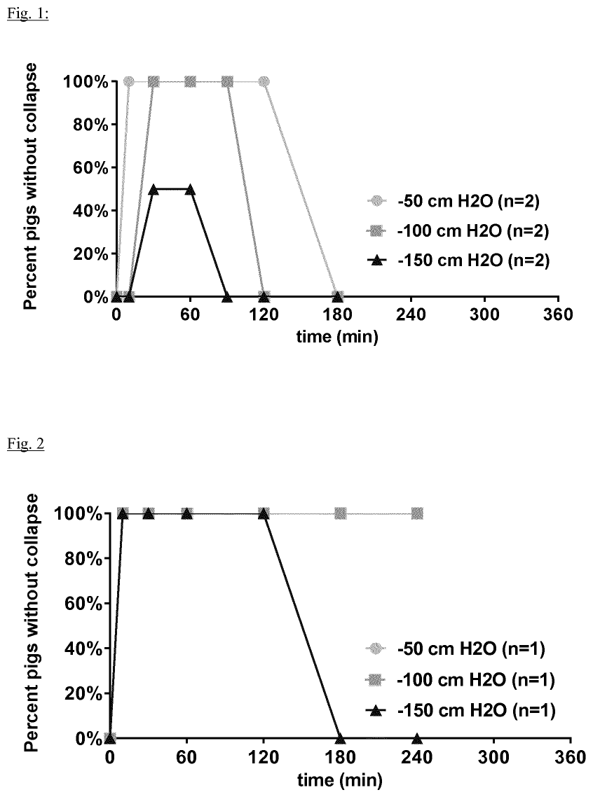

FIG. 1: Effect of i.v. bolus injection of 0.007 mg/kg followed by an i.v. infusion of 0.0025 mg/kg/h for four hours of the [4-(3,4-Dihydroisoquinolin-2(1H)-yl)piperidin-1-yl][2-(2-oxa-6-azaspiro[3.3]hept-6-yl)pyrimidin-5-yl]methanone given at time point 0 min on upper airway collapsibility at different levels of negative pressure. Percentages of pigs with no collapse are given. Mean values.

FIG. 2: Effect of i.v. bolus injection of 0.07 mg/kg followed by an i.v. infusion of 0.025 mg/kg/h for four hours of the [4-(3,4-Dihydroisoquinolin-2(1H)-yl)piperidin-1-yl][2-(2-oxa-6-azaspiro[3.3]hept-6-yl)pyrimidin-5-yl]methanone given at time point 0 min on upper airway collapsibility at different levels of negative pressure. Percentages of pigs with no collapse are given. Mean values.

From the above mentioned data it can be deducted that the α2-Adrenoceptor subtype C (alpha-2C) antagonists of formula (I) are suitable to treat sleep-related breathing disorders, preferably obstructive and central sleep apneas and snoring.

1. A method for the treatment and/or prophylaxis of a sleep-related breathing disorder, comprising administering systematically and/or locally a therapeutically effective amount of at least one compound to a subject in need thereof, wherein the compound is a compound of formula (I): 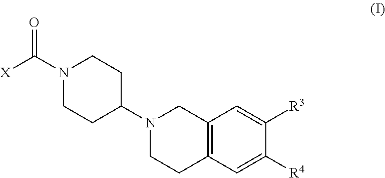
wherein
X is a group 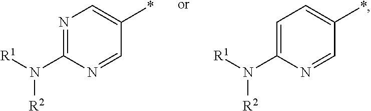
R¹ is C₁-C₆-alkyl or C₃-C₅-cycloalkyl,
wherein alkyl is substituted by 1 to 2 substituents independently of one another selected from the group consisting of hydroxy, C₁-C₄-alkoxy and haloalkoxy
and
R² is hydrogen or C₁-C₄-alkyl,
or
R¹ and R² together with the nitrogen atom to which they are attached form a 4- to 7-membered N-heterocycle,
wherein the N-heterocycle may be substituted by 1 to 3 substituents independently of one another selected from the group consisting of oxo, hydroxy, monofluoromethyl, difluoromethyl, trifluoromethyl, hydroxycarbonyl, tert-butoxycarbonyl, aminocarbonyl, C₁-C₄-alkyl, C₁-C₄-alkoxy, C₁-C₄-alkoxy-C₁-C₄-alkyl, halogen and hydroxyalkyl,
or
wherein the N-heterocycle may have two substituents which, together with the carbon atom of the N-heterocycle to which they are jointly attached, form a 4- to 6-membered heterocycle,
wherein this heterocycle for its part may be substituted by 1 to 3 substituents independently of one another selected from the group consisting of oxo, methyl and ethyl,
R³ is hydrogen, fluorine, methoxy or ethoxy
and
R⁴ is hydrogen, fluorine, methoxy or ethoxy,
or a salt, or a solvate, or a solvate of a salt thereof.

2. The method of claim 1, wherein
R¹ is C₁-C₆-alkyl or C₃-C₅-cycloalkyl,
wherein alkyl is substituted by 1 to 2 substituents independently of one another selected from the group consisting of hydroxy and C₁-C₄-alkoxy,
and
R² is hydrogen or C₁-C₄-alkyl,
or
R¹ and R² together with the nitrogen atom to which they are attached form a 4- to 7-membered N-heterocycle,
wherein the N-heterocycle may be substituted by 1 to 3 substituents independently of one another selected from the group consisting of oxo, hydroxy, monofluoromethyl, difluoromethyl, trifluoromethyl, hydroxycarbonyl, tert-butoxycarbonyl, aminocarbonyl, C₁-C₄-alkyl, C₁-C₄-alkoxy and halogen, or
wherein the N-heterocycle may have two substituents which, together with the carbon atom of the N-heterocycle to which they are jointly attached, form a 4- to 6-membered heterocycle,
wherein this heterocycle for its part may be substituted by 1 to 3 substituents independently of one another selected from the group consisting of oxo, methyl and ethyl,
R³ is hydrogen, fluorine, methoxy or ethoxy,
and
R⁴ is hydrogen, fluorine, methoxy or ethoxy,
or a salt, or a solvate, or a solvate of a salt thereof.

3. The method of claim 1, wherein
R¹ is C₂-C₆-alkyl,
wherein alkyl is substituted by a substituent selected from the group consisting of hydroxy, methoxy and ethoxy,
and
R² is hydrogen,
or
R¹ and R² together with the nitrogen atom to which they are attached form an azetidine, pyrrolidine, piperidine, azepane, piperazine, morpholine, thiomorpholine, 1-oxidothiomorpholine or 1,1-dioxidothiomorpholine,
wherein azetidine, pyrrolidine, piperidine, azepane, piperazine,
morpholine, thiomorpholine, 1-oxidothiomorpholine and 1,1-dioxidothiomorpholine may be substituted by 1 to 2 substituents independently of one another selected from the group consisting of hydroxy, trifluoromethyl, hydroxycarbonyl, C₁-C₃-alkyl, methoxy and methoxymethyl,
or
wherein azetidine, pyrrolidine, piperidine, azepane, piperazine and morpholine may have two substituents which, together with the carbon atom of the azetidine, pyrrolidine, piperidine, azepane, piperazine or morpholine to which they are jointly attached, form an azetidine, oxetane or 1,1-dioxidothietane,
where the azetidine, oxetane or 1,1-dioxidothietane may be substituted by 1 to 2 substituents independently selected from the group consisting of methyl and ethyl,
R³ is hydrogen
and
R⁴ is hydrogen, fluorine or methoxy,
or
R³ is hydrogen, fluorine or methoxy
and
R⁴ is hydrogen,
or a salt, or a solvate, or a solvate of a salt thereof.

4. The method of claim 1, wherein
R¹ is C₂-C₄-alkyl,
wherein alkyl is substituted by a substituent selected from the group consisting of hydroxy and methoxy,
and
R² is hydrogen,
or
R¹ and R² together with the nitrogen atom to which they are attached form an azetidine, pyrrolidine, morpholine or 1,1-dioxidothiomorpholine,
where azetidine, pyrrolidine, morpholine or 1,1-dioxidothiomorpholine may be substituted by 1 to 2 substituents selected independently from the group consisting of hydroxycarbonyl, methyl, trifluoromethyl, methoxy and methoxymethyl,
or
R¹ and R² together with the nitrogen atom to which they are attached form an azetidine, where the azetidine may have two substituents which, together with the carbon atom of the azetidine to which they are jointly attached, form an oxetane or 1,1-dioxidothietane,
R³ is hydrogen, fluorine or methoxy
and
R⁴ is hydrogen,
or
R³ is hydrogen,
and
R⁴ is hydrogen, fluorine or methoxy,
or a salt, or a solvate, or a solvate of a salt thereof.

5. The method of claim 1, wherein
R¹ is C₂-C₄-alkyl,
wherein alkyl is substituted by a substituent selected from the group consisting of hydroxy and methoxy,
and
R² is hydrogen,
or
R¹ and R² together with the nitrogen atom to which they are attached form an azetidine, pyrrolidine, morpholine or 1,1-dioxidothiomorpholine,
wherein azetidine, pyrrolidine, morpholine or 1,1-dioxidothiomorpholine may be substituted by 1 to 2 substituents selected independently from the group consisting of hydroxycarbonyl, methyl, trifluoromethyl, methoxy and methoxymethyl,
or
R¹ and R² together with the nitrogen atom to which they are attached form an azetidine, where the azetidine may have two substituents which, together with the carbon atom of the azetidine to which they are jointly attached, form an oxetane, or 1,1-dioxidothietane
R³ is hydrogen or fluorine,
and
R⁴ is hydrogen, fluorine or methoxy,
or a salt, or a solvate, or a solvate of a salt thereof.

6. The method of claim 1, wherein
X is a group 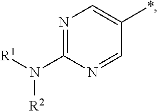
R¹ is C₂-C₄-alkyl,
where alkyl is substituted by a substituent selected from the group consisting of hydroxy and methoxy,
and
R² hydrogen,
or
R¹ and R² together with the nitrogen atom to which they are attached form an azetidine, pyrrolidine, morpholine or 1,1-dioxidothiomorpholine,
wherein azetidine, pyrrolidine, morpholine or 1,1-dioxidothiomorpholine may be substituted by 1 to 2 substituents selected independently from the group consisting of hydroxycarbonyl and methyl,
or
R¹ and R² together with the nitrogen atom to which they are attached form an azetidine,
wherein the azetidine may have two substituents which together with the carbon atom of the azetidine to which they are jointly attached, form an oxetane,
R³ is hydrogen, fluorine or methoxy,
and
R⁴ is hydrogen,
or
R³ is hydrogen,
and
R⁴ is hydrogen, fluorine or methoxy,
or a salt, or a solvate, or a solvate of a salt thereof.

7. The method of claim 6, wherein
R¹ and R² together with the nitrogen atom to which they are attached form an azetidine, wherein the azetidine has two substituents which, together with the carbon atom of the azetidine to which they are jointly attached, form an oxetane,
R³ is hydrogen,
and
R⁴ is hydrogen,
or a salt, or a solvate, or a solvate of a salt thereof.

8. The method of claim 1, wherein the sleep-related breathing disorders are obstructive or central sleep apneas and snoring.

9. The method of claim 1, further comprising administering the at least one compound of formula (I) in combination with one or more other active compounds to the subject.

10. The method of claim 1, wherein the at least one compound of formula (I) is in a pharmaceutical composition and the pharmaceutical composition further comprises one or more inert non-toxic pharmaceutically suitable excipients.

11. The method of claim 1, wherein the at least one compound of formula (I) is in a pharmaceutical composition comprising a combination of the at least one compound of formula (I), one or more other active compounds, and one or more inert non-toxic pharmaceutically suitable excipients.

12. The method of claim 1, wherein the at least one compound of formula (I) is in a pharmaceutical composition and the pharmaceutical composition further comprises an inert, non-toxic, pharmaceutically acceptable additive.

13. The method according to claim 12, wherein the method further comprises administering systemically and/or locally at least one further active compound selected from the group consisting of muscarinic receptor antagonists, mineralocorticoid receptor antagonists, diuretics, and corticosteroids to the subject.

14. The method of claim 1, further comprising administering the at least one compound of formula (I) with one or more further active ingredients selected from the group consisting of muscarinic receptor antagonists, mineralocorticoid receptor antagonists, diuretics, and corticosteroids to the subject.
